(19) United States
(12) Patent Application Publication
(10) Pub. No.: US 2009/0098214 A1
(43) Pub. Date: Apr. 16, 2009

(54) SKIN-REVITALIZING COSMETIC COMPOSITION

(75) Inventor: Teruyuki Nanbu, Hakata-ku (JP)

Correspondence Address: OBLON, SPIVAK, MCCLELLAND MAIER & NEUSTADT, P.C., 1940 DUKE STREET, ALEXANDRIA, VA, 22314, US

(73) Assignee: ASKA CORPORATION Co., Ltd., Hakata-ku (JP)

(21) Appl. No.: 12/051,987

(22) Filed: Mar. 20, 2008

(30) Foreign Application Priority Data
Oct. 10, 2007 (JP) 2007-263907

(51) Int. Cl. A61K 35/50; A61K 36/886; A61K 38/39; A61P 17/00

(52) U.S. Cl. 424/583; 424/744; 514/12

(57) ABSTRACT

The invention provides a skin-revitalizing cosmetic composition for alleviating or eliminating skin problems such as dry skin, wrinkles, and age spots, the composition containing a skin-revitalizing agent at high concentration. The skin-revitalizing cosmetic composition contains a skin-revitalizing agent selected from the group consisting of aloe extract, placenta extract, hydrolyzed elastin, hydrolyzed collagen, and sodium hyaluronate, wherein the skin-revitalizing agent is in the form of nanoparticles obtained through a nanoparticle-forming treatment.

### BACKGROUND OF THE INVENTION

1. Field of the Invention

The present invention relates to a cosmetic composition, and more particularly to a skin-revitalizing cosmetic composition for alleviating or eliminating skin problems such as dry skin, wrinkles, and age spots.

2. Background Art

There have been well known cosmetic products containing a skin-revitalizing agent for alleviating or eliminating skin problems such as dry skin, wrinkles, and age spots. For example, Japanese Patent Application Laid-Open (kokai) No. 2005-53842 discloses a cosmetic product containing an aloe extract; Japanese Patent Application Laid-Open (kokai) No. 2001-72567 discloses a cosmetic product containing a placenta extract; Japanese Patent Application Laid-Open (kokai) No. 2006-131557 discloses a cosmetic product containing hydrolyzed elastin; Japanese Patent Application Laid-Open (kokai) No. 2003-300854 discloses a cosmetic product containing hydrolyzed collagen; and Japanese Patent Application Laid-Open (kokai) No. H05-345717 discloses a cosmetic product containing sodium hyaluronate.

However, such a skin-revitalizing agent is difficult to dissolve in water, and thus can only be incorporated into a currently employed cosmetic product in an amount of up to 1 wt. % at most. Therefore, a cosmetic product containing such a skin-revitalizing agent fails to satisfactorily exhibit the effect of the agent.

### SUMMARY OF THE INVENTION

In view of the foregoing, an object of the present invention is to provide a cosmetic composition which can contain such a skin-revitalizing agent at a high concentration, thereby attaining an improved skin-revitalizing effect.

The present inventors have conducted extensive studies on the aforementioned skin-revitalizing agents, and as a result have found that when a skin-revitalizing agent is subjected to a treatment for forming nanoparticles (hereinafter the treatment may be referred to as a “nanoparticle-forming treatment”), and then the thus-formed nanoparticles of the agent are incorporated into a cosmetic composition, the agent concentration of the cosmetic composition can be increased to more than 1 wt. %. The present invention has been accomplished on the basis of this finding.

Accordingly, the present invention provides a skin-revitalizing cosmetic composition containing a skin-revitalizing agent selected from the group consisting of aloe extract, placenta extract, hydrolyzed elastin, hydrolyzed collagen, and sodium hyaluronate, wherein the skin-revitalizing agent is in the form of nanoparticles obtained through a nanoparticle-forming treatment.

According to the present invention, since a skin-revitalizing agent can be incorporated into a cosmetic composition at a high concentration, a further improved skin-revitalizing effect of the composition can be obtained.

### DETAILED DESCRIPTION OF PREFERRED EMBODIMENTS

The present invention will next be described in more detail.

The skin-revitalizing cosmetic composition of the present invention contains a skin-revitalizing agent selected from the group consisting of aloe extract, placenta extract, hydrolyzed elastin, hydrolyzed collagen, and sodium hyaluronate, wherein the skin-revitalizing agent is in the form of nanoparticles obtained through a nanoparticle-forming treatment.

Such a skin-revitalizing agent itself is well known in the art, and thus detailed descriptions thereof are omitted.

In the present invention, such a skin-revitalizing agent is subjected to a nanoparticle-forming treatment, and then incorporated into a cosmetic composition. The nanoparticle-forming treatment of a skin-revitalizing agent may be carried out by means of a commercially available Nanomizer (e.g., Nanomaker, product of Nanomax).

Specifically, when a skin-revitalizing agent is dispersed in water, and the resultant aqueous dispersion is subjected to a nanoparticle-forming treatment, the skin-revitalizing agent can be formed into nanoparticles having a size (diameter) of 10 to 150 nm. The thus-formed nanoparticles of the skin-revitalizing agent can be incorporated into an aqueous cosmetic composition at a high concentration; i.e., more than 1 wt. % (generally more than 1 wt. % and up to 10 wt. %).

The skin-revitalizing cosmetic composition of the present invention which contains, as an essential component, nanoparticles of a skin-revitalizing agent formed through the aforementioned treatment may also contain additional ingredients as additives. Examples of the additives include ceramide and royal jelly extract.

### EXAMPLES

The present invention will next be described by way of examples, which should not be construed as limiting the invention thereto.

Each of the skin-revitalizing agents shown in Table 1 was incorporated into purified water in an amount (wt. %) shown therein. In the Comparative Examples, each of the skin-revitalizing agents shown in Table 1 was added to purified water in an amount shown therein, followed by stirring at 50° C. for 30 minutes and then cooling to 20° C. Immediately thereafter, the state of dissolution was observed (shown in the column “Immediately after dissolution” of Table 1). Subsequently, the sample was stored at 5° C. for 24 hours, and the state of dissolution was also observed (shown in the column “After storage” of Table 1).

In the Examples, each of the skin-revitalizing agents shown in Table 1 was added to purified water in an amount shown therein, followed by stirring at 50° C. for 30 minutes. The resultant aqueous dispersion was subjected to a nanoparticle-forming treatment by means of Nanomaker (product of Nanomax). The thus-formed nanoparticles of the skin-revitalizing agent were found to have a size of 80 nm. Thereafter, the thus-treated dispersion was cooled to 20° C. Immediately thereafter, the state of dissolution was observed (shown in the column “Immediately after dissolution” of Table 1). Subsequently, the sample was stored at 5° C. for 24 hours, and the state of dissolution was also observed (shown in the column “After storage” of Table 1).

TABLE 1

| Skin- | | Comparative Examples | | Examples | |
|---|---|---|---|---|---|
| revitalizing | | Immediately | | Immediately | |
| agent | Amount | after dissolution | After storage | after dissolution | After storage |
| Aloe extract | 1% | Dissolved | Dissolved | Dissolved | Dissolved |
| | 5% | Dissolved | Dissolved | Dissolved | Dissolved |
| | 10% | Dissolved | Precipitation | Dissolved | Dissolved |
| | | | of crystals | | |
| | 15% | Not dissolved | Precipitation | Dissolved | Dissolved |
| | | | of crystals | | |
| Placenta | 1% | Dissolved | Dissolved | Dissolved | Dissolved |
| extract | 5% | Dissolved | Precipitation | Dissolved | Dissolved |
| | | | of crystals | | |
| | 10% | Dissolved | Precipitation | Dissolved | Dissolved |
| | | | of crystals | | |
| | 15% | Not dissolved | Precipitation | Dissolved | Dissolved |
| | | | of crystals | | |
| Hydrolyzed | 1% | Dissolved | Dissolved | Dissolved | Dissolved |
| elastin | 5% | Dissolved | Dissolved | Dissolved | Dissolved |
| | 10% | Dissolved | Precipitation | Dissolved | Dissolved |
| | | | of crystals | | |
| | 15% | Dissolved | Precipitation | Dissolved | Dissolved |
| | | | of crystals | | |
| Hydrolyzed | 1% | Dissolved | Dissolved | Dissolved | Dissolved |
| collagen | 5% | Dissolved | Dissolved | Dissolved | Dissolved |
| | 10% | Dissolved | Dissolved | Dissolved | Dissolved |
| | 15% | Dissolved | Precipitation | Dissolved | Dissolved |
| | | | of crystals | | |
| Sodium | 0.5% | Dissolved | Dissolved | Dissolved | Dissolved |
| hyaluronate | 1% | Dissolved | Dissolved | Dissolved | Dissolved |
| | 2% | Not dissolved | Not dissolved | Dissolved | Dissolved |

As is clear from the data shown in Table 1, the cosmetic compositions of the present invention, which contain nanoparticles of a skin-revitalizing agent, maintained a dissolved state even when they had high skin-revitalizing agent concentrations.

As described above, the cosmetic composition of the present invention is suitable for use as a skin-revitalizer for alleviating or eliminating skin problems such as dry skin, wrinkles, and age spots.

What is claimed is:

1. A skin-revitalizing cosmetic composition containing a skin-revitalizing agent selected from the group consisting of aloe extract, placenta extract, hydrolyzed elastin, hydrolyzed collagen, and sodium hyaluronate, wherein the skin-revitalizing agent is in the form of nanoparticles obtained through a nanoparticle-forming treatment.

2. A skin-revitalizing cosmetic composition as described in claim 1, which contains the skin-revitalizing agent at a concentration of more than 1 wt. % and up to 10 wt. %.
